SEQUENCE LISTING (1 sequence)

SEQ ID NO: 1 - protein, 219 aa, Homo sapiens
MLRAGAPTGDLPRAGEVHTGTTIMAVEFDGGVVMGSDSRVSAGEAVVNRVFDKLSPLHERIYCALSGSAADAQAVADMAAYQLELHGIELEEPPLVLAAANVVRNISYKYREDLSAHLMVAGWDQREGGQVYGTLGGMLTRQPFAIGGSGSTFIYGYVDAAYKPGMSPEECRRFTTDAIALAMSRDGSSGGVIYLVTITAAGVDHRVILGNELPKFYDE

(19) United States
(12) Patent Application Publication
(10) Pub. No.: US 2023/0228757 A1
(43) Pub. Date: Jul. 20, 2023

(54) METHOD FOR JUDGING RISK OF SUFFERING KIDNEY CANCER

(71) Applicants: CHANG GUNG UNIVERSITY, TAOYUAN CITY (TW); CHANG GUNG MEMORIAL HOSPITAL, LINKOU, TAOYUAN CITY (TW)

(72) Inventors: YI-TING CHEN, TAOYUAN CITY (TW); RONG-SHING CHANG, TAOYUAN CITY (TW); YU CHANG, TAOYUAN CITY (TW); CHIEN-LUN CHEN, TAOYUAN CITY (TW); KAI-JIE YU, TAOYUAN CITY (TW); YING-HSU CHANG, TAOYUAN CITY (TW)

(21) Appl. No.: 17/810,798

(22) Filed: Jul. 5, 2022

(30) Foreign Application Priority Data
Jan. 20, 2022 (TW) 111102456

(51) Int. Cl. G01N 33/574; G01N 33/68

(52) U.S. Cl. CPC G01N 33/57438; G01N 33/6872; G01N 2800/52

(57) ABSTRACT

The invention relates to a method for judging risk of suffering kidney cancer. The method includes detecting a PSMB9 protein concentration value from a test sample. And a risk factor value is obtained after processing the PSMB9 protein concentration value with an age. When the score of the risk factor value is greater than a defined threshold value of the risk factor threshold value, then the test sample is classified as a high risk sample of a of potential kidney cancer patient.

### FIELD OF THE INVENTION

This invention is a judging method, particularly a method for judging the risk of suffering kidney cancer.

### SEQUENCE LISTING INCORPORATION BY REFERENCE

The Sequence Listing written in file “3315-1297-METHOD FOR JUDGING RISK OF SUFFERING KIDNEY CANCER.xml” created on Jul. 5, 2022, file size 2,128 bytes, is hereby incorporated by reference in its entirety for all purposes.

### BACKGROUND OF THE INVENTION

Nowadays, thanks for the advancement of modern medicine and the improvement of sanitation and nutritional conditions, the average expected human lifetime has increased significantly compared. Meanwhile, patients that had the short lifetime due to neglecting diseases in the early days expected not treated in time has gradually become the spotlight and even becomes the main cause of severe illness or death.

The common main lethal diseases include malignant tumor (cancer), heart disease, pneumonia, cerebrovascular disease, diabetes, hypertensive disease, chronic lower respiratory tract disease, nephritis nephropathy syndrome and nephropathy, chronic liver disease and liver cirrhosis etc., Among them, the percentage of malignant tumors is the highest one among all causes of death and has become a key item of medical research; the cumulative amount of related research results and treatment methods are extremely considerable.

Not all tumors will develop into malignant tumors. In some cases, tumors formed by abnormal cell proliferation will not invade other parts of the body and are called benign tumors. Malignant tumors, also known as cancers, not only divide in an uncontrollable way, but also locally invade surrounding normal tissues and even metastasize to other parts of the body via the circulatory system or lymphatic system of the body.

Currently, the classification of cancers is based on the cell type of tumor-derived tissue and its biological behavior. In Taiwan, the most common types of cancers include trachea, bronchus and lung cancer, liver and intrahepatic bile duct cancer, colon, rectal and anal cancer, and female breast cancer, etc. The symptoms of different types of cytopathies are different.

Kidney is one of the important organs of the body; its main function is to synthesize urine, excrete toxins and regulate body chemical balance etc. Kidney cancer is a malignant tumor that occurs in kidneys. Kidney tumors are often ignored by patients because they have no obvious symptoms in the early stage. At the time that symptoms such as hematuria, back pain and abdominal mass appear, most of the cases are in the late stage of kidney cancers.

Kidney cancers are mainly divided into renal cell carcinoma and renal pelvis carcinoma; the renal cell carcinoma (RCC) is more common, accounting for about 85% to 90% of kidney cancers. Renal cell carcinoma mainly originates from the epidermis of proximal renal tubules in the kidney. It is known that the main risk factors for RCC are smoking, alcohol drinking and obesity. The percentage of genetic factors is small, and it is more common in male kidneys about ages within forty to seventy years old. In addition, according to statistics, the incidence of kidney cancers (KC) is listed in the top 20 in the statistical ranking of cancer types for both men and women, suffering the renal cell carcinoma prevails.

For the diagnosis of kidney cancer, the current method is to propose evidence on imaging first, and then further confirm it with pathological section result. Yet, about 20% of such early diagnosis by imaging will misdiagnose tumors in the kidney as malignant tumors, resulting in unnecessary follow-up invasive tests.

To reduce the invasiveness of tests and the misjudgment rate, if we can apply the detection of early disease in a non-invasive way, it will help the deficiencies in the current clinical detection. The research and development of non-invasive detection methods is the problem that the technical personnel in the field want to solve thereof.

### SUMMARY

This invention proposes an innovative idea that after the samples of kidney cancer and adjacent normal tissue are processed, the protein components contained in each sample are hydrolyzed and qualitatively and quantitatively detected to find out that a protein named PSMB9 is present in kidney cancer cells. The expression level of PSMB9 protein is higher than that in adjacent normal tissues; the PSMB9 protein is a protein molecule with the amino acid sequence of SEQ ID NO: 1. This test result confirms that the content of PSMB9 protein in the test sample can be used as an effective indicator for early judgment of kidney cancer risk. The PSMB9 values of 39 kidney cancer and hernia patients were used to obtain how much protein concentration was contained in urine, and the ROC curve was combined with Youden's index (J) to find the best tangent point. Then, the threshold value of the risk factor were calculated from this data which calculated the PSMB9 risk score, 0.037 (fmol/μg urine protein).

To achieve the aforesaid goal, this invention reveals a method for judging a risk of suffering kidney cancer based on the aforesaid study results; include the steps of: obtaining a test sample; detecting a PSMB9 protein in the test sample to obtain a PSMB9 protein concentration value, in which the PSMB9 protein has the amino acid sequence of SEQ ID NO: 1; the PSMB9 protein concentration value to obtain a value of a risk factor; comparing the value of the risk factor with a threshold value of the risk factor; and when the value of the risk factor is greater than the threshold value of the risk factor, judge that the test sample has a high risk of suffering kidney cancer; wherein the threshold value of the risk factor is 0.037 (fmol PSMB9 /μg urine protein).

To achieve the aforesaid goal, this invention reveals a method for judging a risk of suffering kidney cancer based on the aforesaid study results; include the steps of: obtaining a test sample; detecting a PSMB9 protein in the test sample to obtain a PSMB9 protein concentration value, in which the PSMB9 protein has the amino acid sequence of SEQ ID NO: 1; multiplying the PSMB9 protein concentration value by an age to obtain a value of a first risk factor; comparing the value of the first risk factor with a threshold value of the first risk factor; when the value of the first risk factor is greater than the threshold value of the first risk factor, judge that the test sample has a high risk of suffering kidney cancer. Wherein the threshold value of the first risk factor is 2.687 (fmol/μg*age).

To achieve the aforesaid goal, this invention reveals another method for judging a risk of suffering kidney cancer, include the steps of: Obtaining a test sample; detecting a PSMB9 protein in the test sample to obtain a PSMB9 protein concentration value, in which the PSMB9 protein has the amino acid sequence of SEQ ID NO: 1; dividing the PSMB9 protein concentration value by an age to obtain a value of a second risk factor; comparing the value of the second risk factor with a threshold value of the second risk factor; and when the value of the second risk factor is greater than the threshold value of the second risk factor, judge that the test sample has a high risk of suffering kidney cancer. Wherein the threshold value of the second risk factor is 0.00032 [(fmol/μg)/age].

In an embodiment, the test sample is the body fluid of a test subject or a normal person, or an extract derived from cells and tissues. Among them, the body fluid is blood or urine, preferably urine. In an embodiment, the testing mode of test sample can be a common protein detection technology. In a specific embodiment, the test sample is subjected to protein concentration and desalting from urine and use trypsin to hydrolyze protein into peptides; then, use an ultrahigh-pressure liquid chromatograph (UPLC) waters for elution; next, use QTRAP® 6500+ LC-MS/MS System to analyze the test sample. In another specific embodiment, the test sample performs immunohistochemistry (IHC) after the biopsy of renal tumor tissue; after staining with an anti-proteasome 20S LMP2 antibody (ab3328), interpret and analyze the biopsy samples.

In an embodiment, make statistics on the method for judging a risk of suffering kidney cancer and obtain the concrete steps of risk factor value as follows: Carry out three repetitive monitoring of “multiple reaction method” experiment on each test sample, confirm the reproducibility of the experiment, obtain the ratio of the light peak area to the heavy peak area on each test sample, and sum up the three repetitive ratios of the same sample and divide the sum by three to calculate the average value, which is used as the relative value of the individual protein test sample. Signals not measured from the sample will also incorporate the data into the calculation. The protein's relative values are summed and divided by the total number of individual test samples to calculate the overall average relative value of each protein in the test sample.

This invention can be used as an early judgment of the risk of kidney cancer in the perspective of achieving the effect of early prevention and treatment and can be used as a reference basis for evaluating the therapeutic outcome after kidney cancer surgery.

To achieve the goal of evaluating the aforesaid kidney cancer post-operatively, this Invention reveals a method for judging a risk of suffering kidney cancer, include the steps of: Obtaining a post-operative test sample, the post-operative test sample is taken from a test subject that has undergone a medicine treatment or a surgical treatment or a combination thereof; detecting a PSMB9 protein in the post-operative test sample to obtain a PSMB9 protein concentration value, in which the PSMB9 protein has the amino acid sequence of SEQ ID NO: 1; multiplying the PSMB9 protein concentration value by an age to obtain a value of a third risk factor; comparing the value of the third risk factor with a threshold value of the third risk factor; when the value of the third risk factor is greater than the threshold value of the third risk factor, judge that the post-operative test sample has a high risk of suffering kidney cancer. Wherein the post-operative test sample is the urine from a test subject, and the threshold value of the third risk factor is 2.687 (fmol/μg*age).

To achieve the goal of the aforesaid kidney cancer post-operative evaluation, this invention reveals another method for judging a risk of suffering kidney cancer, include the steps of: Obtaining a post-operative test sample, the post-operative test sample is taken from a test subject that has undergone a medicine treatment or a surgical treatment or a combination of both; detecting a PSMB9 protein in the post-operative test sample to obtain a PSMB9 protein concentration value, in which the PSMB9 protein has the amine group of SEQ ID NO: 1 acid sequence; dividing the PSMB9 protein value by an age of the source patient to obtain a value of a fourth risk factor; comparing the value of the fourth risk factor with a threshold value of the fourth risk factor; when the value of the fourth risk factor is greater than the threshold value of the fourth risk factor, judge that the post-operative test sample has a high risk of suffering kidney cancer; wherein the post-operative test sample is the urine of a test subject, and the threshold value of the fourth risk factor is 0.00032 [(fmol/μg)/age].

Since the PSMB9 protein is an immune-related protein subunit, when the kidney has an inflammatory response, the expression level of the PSMB9 protein is also up-regulated. Therefore, when using the PSMB9 protein as a kidney cancer risk assessment index; to test the PSMB9 protein, it is preferable to exclude the possibility that is under kidney inflammation. Therefore, in an embodiment of this invention, for the method for judging a risk of suffering kidney cancer, in the step of obtaining a test sample (or obtaining a post-operative test sample), it further includes the step of: Detecting a leukocyte of the test sample/the post-operative test sample to obtain a leukocyte number value; comparing the leukocyte number value and a leukocyte number threshold value; when the leukocyte number value is less than the leukocyte number threshold value, continue to detect the PSMB9 protein; wherein the leukocyte number threshold value is 30/HPF, and the leukocyte number value is obtained by a general precipitation method or a conventional detection method.

### BRIEF DESCRIPTION OF THE DRAWINGS

FIG. 1: The step flowchart in the first embodiment of this invention;

FIG. 2A: The relative expression result chart for the PSMB9 protein multiplies the age in the first embodiment of this invention;

FIG. 2B: The ROC curve analysis chart for the PSMB9 protein multiplies the age in the first embodiment of this invention;

FIG. 3: The step flowchart in the second embodiment of this invention;

FIG. 4A: The relative expression result chart for the PSMB9 protein divided by the age in the second embodiment of this invention;

FIG. 4B: The ROC curve analysis chart for the PSMB9 protein divided by the age in the second embodiment of this invention;

FIG. 5: The step flowchart in the third embodiment of this invention;

FIG. 6: The step flowchart in the fourth embodiment of this invention;

FIG. 7A: The flowchart of the test steps collocating leukocytes detection in the first embodiment of this invention;

FIG. 7B: The flowchart of the test steps collocating leukocytes detection in the second embodiment of this invention;

FIG. 8A: The flowchart of the test steps collocating leukocytes detection in the third embodiment of this invention; and

FIG. 8B: The flowchart of the test steps collocating leukocytes detection in the fourth embodiment of this invention.

### DETAILED DESCRIPTION

To enable the review committee members to have a deeper realization and understanding of the features and functions of this invention, we hereby put the embodiment and detailed explanation in below:

Due to the fact that only relying on regular health checks including kidney ultrasound and urine examination can have the opportunity to find early kidney cancer; however, the general domestic residents do not have the habit of taking regular health checks, and most of the kidney cancer patients are in the late stage when they are found suffering from kidney cancer and thus have missed the opportunity for cure. From this reason, this invention proposes a method of judging the risk of suffering kidney cancer to solve the problems caused by the conventional technology.

The method of judging the risk of suffering kidney cancer of this invention, the characteristics, and the cooperated structure and method it comprises will be further described below:

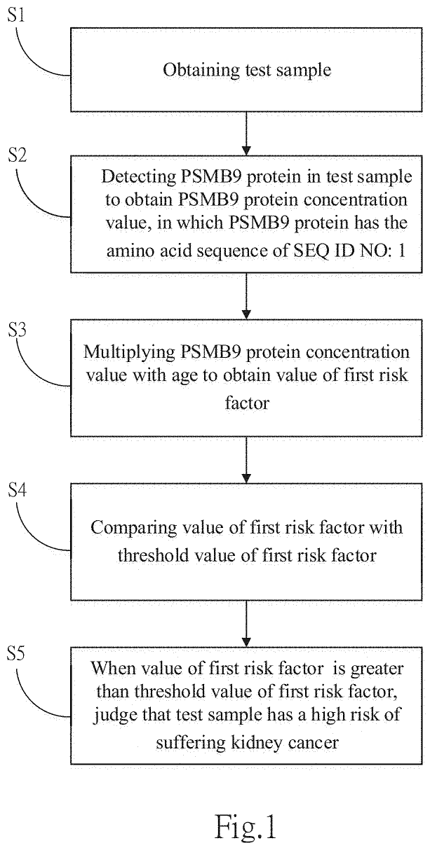

Referring to FIG. 1, it is the step flowchart in the first embodiment of this invention; as shown in the figure, there is a method of judging the risk of suffering kidney cancer, the steps comprises:

S1: Obtaining test sample;

S2: Detecting PSMB9 protein in test sample to obtain PSMB9 protein concentration value, in which PSMB9 protein has the amino acid sequence of SEQ ID NO: 1;

S3: Multiplying PSMB9 protein concentration value with age to obtain value of first risk factor;

S4: Comparing value of first risk factor with threshold value of first risk factor; and

S5: When value of first risk factor is greater than threshold value of first risk factor, judge that test sample has a high risk of suffering kidney cancer.

As shown in step S1, it should obtain a test sample before performing the test, and the test sample can be the body fluid of a test subject or a normal person, or an extract derived from cells and tissues. Among them, the body fluid is blood or urine. The urine sample is obtained from surgical patients at Linkou Chang Gung Memorial Hospital (Taoyuan City, Taiwan) and had passed the Institutional Review Board (IRB) and signed the consent form for the collection of human samples before receiving the samples (no gender restriction on test subjects).

In the first embodiment of this Invention, the processing method of using urine as the test sample is: Adding 100 μl of protease inhibitor (cOmplete™, EDTA-free Protease Inhibitor Cocktail, Roche, Germany) to every 50 ml of urine; dissolved in 500 μl secondary deionized water, and mixed with 100 μl of sodiumazide (NaN₃, Sigma, 71289-5G, USA); then, using a floor-standing refrigerated centrifuge (Universal Refrigerated Centrifuge Model 5922, KUBOTA, P71304-B600, Japan) to centrifuge at 5000 g for 30 minutes at 4° C.; leave the supernatant, keep the supernatant, pour 12.5 ml of urine into a 10 kDacut-off concentrating centrifuge tube (Amicon™ Ultra-1510KCentrifugal Filter Units, R7JA29943, Ireland) and centrifuge at 5000 g for 30 minutes at 4° C.; discard the precipitate after the end of centrifuge.

Wash off the salts with 12.5 ml of 20% acetonitrile (ACN, J. T. Baker, 9017-03, U.S.); centrifuge for 30 minutes under the same conditions and discard the lower subnatant afterward; then, add 12 ml of secondary deionized water twice, centrifuge under the same conditions for 30 minutes and discard the precipitate each time. Finally, pour the concentrated urine on the membrane and rinse the edge of membrane with 200 μl secondary deionized water to wash out the remaining proteins, and combine the urine proteins on the membrane and collect them into a tube.

Yet, if tissue fragments are used as test samples, the processing method is as follows: First, take out about 20 μg of tissue fragments and add 200 μl of 0.1% RapiGest SF Surfcant (Waters, USA), and utilize Precellys24 homogenizer (Bertin Instrument, France) to shake for 10 seconds at 6500 rpm and repeat for three times; after standing the sample on ice for 5 minutes, repeat the above shaking step and then collect the supernatant. Next, centrifuge the sample at 13,000 rpm for 20 minutes at 4° C., remove the precipitate, and store the supernatant at −80° C. for subsequent use.

Continuing as shown in step S2; detect a PSMB9 protein in the test sample to obtain a PSMB9 protein concentration value; the PSMB9 protein concentration value is measured by a liquid chromatography/multiple reaction monitoring mass spectrometer; this PSMB9 protein has the amino acid sequence of SEQ ID NO: 1; the SEQ ID NO: 1 is: MLRAGAPTGDLPRAGEVHTGTTIMAVEFDGGVVMGSDSRVSAGEAVVNRVFDKLSPLHE RIYCALSGSAADAQAVADMAAYQLELHGIELEEPPLVLAAANVVRNISYKYREDLSAHLM VAGWDQREGGQVYGTLGGMLTRQPFAIGGSGSTFIYGYVDAAYKPGMSPEECRRFTTDAI ALAMSRDGSSGGVIYLVTITAAGVDHRVILGNELPKFYDE.

In step S2, first, determine the content of the PSMB9 protein in urine by bicinchoninic acid (BCA, Pierce™ BCA Protein Assay Kit, QH220532A). Prepare the standard sample (take the original solution (2 mg/ml) and dilute it into 0, 0.25, 0.5, 0.75, 1, 1.5 mg/ml BSA); the diluted concentration of samples should be within the range of 1 to 10 times of the standard curve; add the samples to the 96-well plates sequentially. The experiment was repeated for three times; add 10 μl sample and 200 μl reagent A+reagent B mixer (A:B=50:1) into each of the sample tanks, making reaction in the shaking incubator (LM-400 YIH DER) at 37° C. and shaking 90 rpm in the dark for 30 minutes. Finally, using an Enzyme-linked immunosorbent assay reader to measure the absorbance (wavelength 562 nm) by selecting the BCA assay protein quantitative mode; and calculate the protein concentration of the sample.

This PSMB9 protein concentration value is measured by this liquid chromatography/multiple reaction monitoring mass spectrometer. The mass spectrometer system used in this invention is a quadrupole ion trap mass analyzer QTRAP® 6500+ LC-MS/MS System (SCIEX), and the method was developed using Skyline software (MacCoss Lab Software).

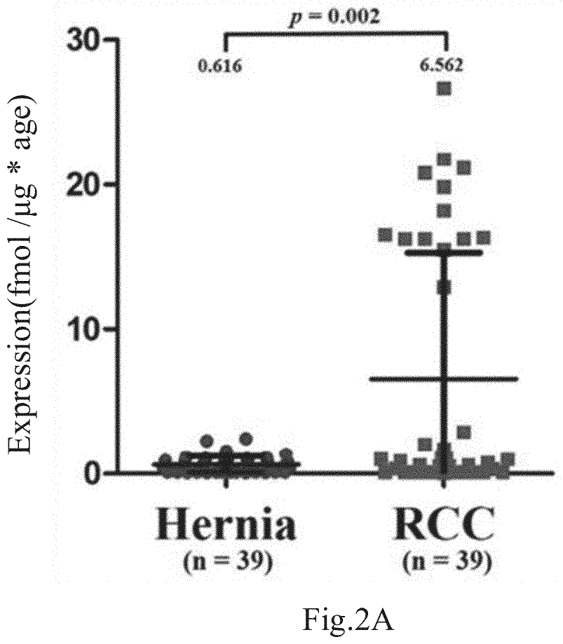

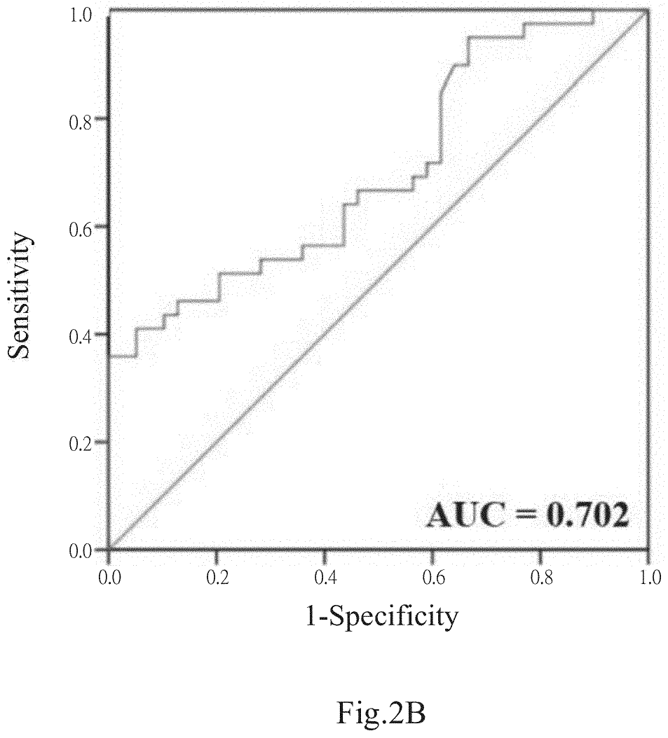

Continuing in step S3, taking the PSMB9 protein concentration values (fmol of the PSMB9 protein contained in each μg of urine) of urine samples by LC-MRM/MS obtained from thirty-nine (n=39) kidney cancer (RCC) and hernia patients; and making calculation along with the ROC curve and Youden's index (J). Have the PSMB9 protein concentration value multiply an age (the age of the test subject) to obtain a value of a first risk factor (fmol/μg*age), the results of which are shown in FIGS. 2A and 2B, which are the relative expression result chart for the PSMB9 protein multiplies the age in the first embodiment of this Invention and the ROC curve analysis chart for the PSMB9 protein multiplies the age in the first embodiment of this Invention. From the figures it can be seen that the expression levels (the value of the first risk factor) of the test sample of kidney cancer are way higher than that of non-kidney cancer (fold change, FC=10.65); when the threshold was set to 2.687 (fmol/μg*age), the AUC value was 0.702, the sensitivity was 0.359, and the specificity was 1.

Continuing as shown in step S4, take the aforesaid threshold 2.687 (fmol/μg*age) as a threshold value of the first risk factor (fmol/μg*age), and compare the value of the first risk factor of the test sample with the threshold value of the first risk factor.

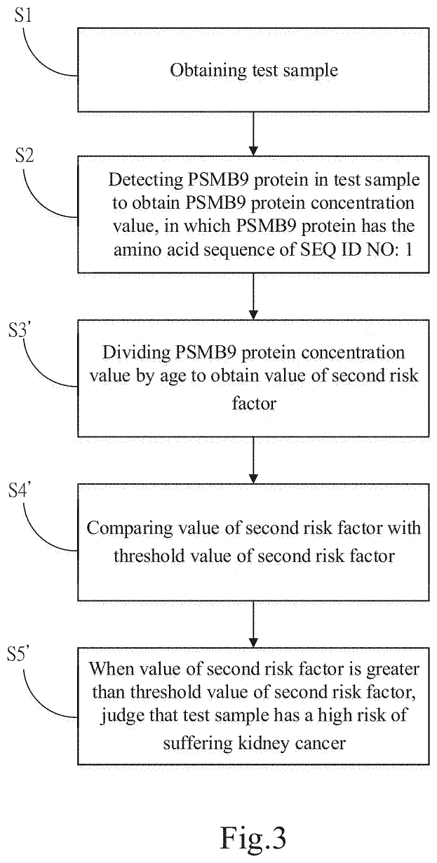

Finally, as shown in step S5, when the value of the first risk factor is greater than the threshold value of the first risk factor, it is judged that the test sample is a high risk of suffering kidney cancer. Referring to FIG. 3, which is the step flowchart in the second embodiment of this Invention. As shown in the figure, there is a method of judging the risk of suffering kidney cancer, the steps comprises:

S1: Obtaining test sample;

S2: Detecting PSMB9 protein in test sample to obtain PSMB9 protein concentration value, in which PSMB9 protein has the amino acid sequence of SEQ ID NO: 1;

S3′: Dividing PSMB9 protein concentration value by age to obtain value of second risk factor;

S4′: Comparing value of second risk factor with threshold value of second risk factor; and

S5′: When value of second risk factor is greater than threshold value of second risk factor, judge that test sample has a high risk of suffering kidney cancer.

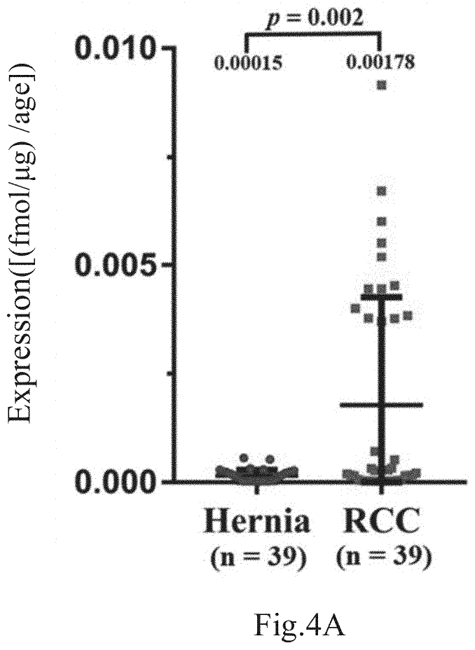

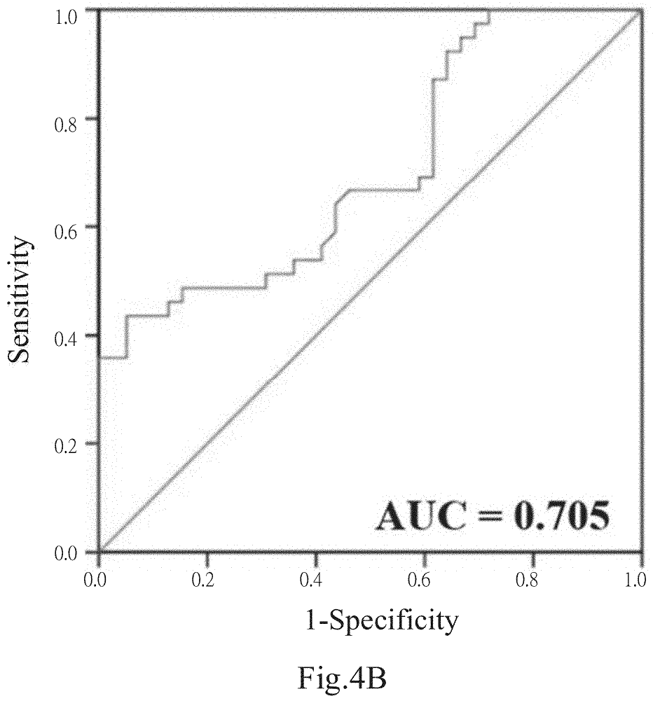

Steps 1 and 2 of the second embodiment of this invention are identical to the first embodiment of this invention; the difference is that by following step S3′, urine samples were obtained by LC-MRM/MS from thirty-nine (n=39) kidney cancer (RCC) and hernia patients to obtain the PSMB9 protein concentration (fmol of PSMB9 protein per pg urine); and making calculations along with the ROC curve and Youden's index (J). Dividing the PSMB9 protein concentration value by the age (the age of the test subject) to obtain a value of the second risk factor [(fmol/μg)/age], the results are shown in FIGS. 4A and 4B, they are the relative expression result chart for the PSMB9 protein divided by the age in the second embodiment of this Invention and the ROC curve analysis chart for the PSMB9 protein divided by the age in the second embodiment of this invention. From the figures, we can see that the expression levels (the value of the second risk factor) of the test sample of kidney cancer are way higher than that of non-kidney cancer samples (fold change, FC=11.72). When the threshold was set to 0.00032 [(fmol/μg)/age], the AUC value was 0.705, the sensitivity was 0.436, and the specificity was 0.949.

Also, as shown in step S4′, take the threshold 0.00032 [(fmol/μg)/age] shown above as a threshold value of the second risk factor, and compare the value of the second risk factor of the test sample with the threshold value of the second risk factor. Finally, as shown in step S5′, if the value of the second risk factor is greater than the threshold value of the second risk factor, it is judged that the test sample has a high risk of suffering kidney cancer.

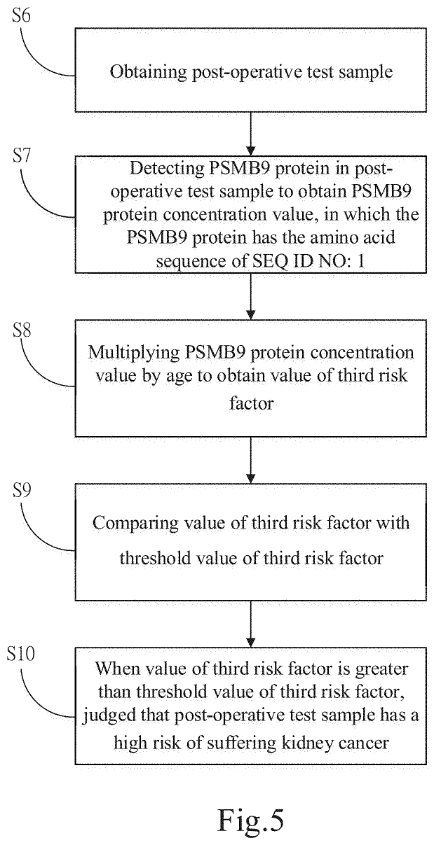

Referring to FIG. 5, it is the step flowchart in the third embodiment of this Invention. As shown in the figure, there is a method of judging the risk of suffering kidney cancer, the step comprises:

S6: Obtaining post-operative test sample;

S7: Detecting PSMB9 protein in post-operative test sample to obtain PSMB9 protein concentration value, in which the PSMB9 protein has the amino acid sequence of SEQ ID NO: 1;

S8: Multiplying PSMB9 protein concentration value by age to obtain value of third risk factor;

S9: Comparing value of third risk factor with threshold value of third risk factor; and

S10: When value of third risk factor is greater than threshold value of third risk factor, judged that post-operative test sample has a high risk of suffering kidney cancer.

The third embodiment of this invention is used as the method of judging the risk of suffering kidney cancer after surgery; the steps are identical to the first embodiment, in which a threshold value of a third risk factor of the third embodiment is 2.687 (fmol/μg*age). Another difference is that the sample obtained in the third embodiment of this invention is a post-operative test sample; of which the test subject has undergone a surgical treatment or a medicine treatment or a combination of both. After having undergone the surgical treatment, the method of judging the risk of suffering kidney cancer can be used to further judge how effective the surgical treatment is on the test subject.

Except simply figuring out the effect of carrying out the surgical treatment, it can also be applied to the effect of simply using the medicine treatment and can be further figure out that the effect of medicine treatment being further carried out after the surgical treatment (cooperative treatment because of the tumor transfer).

The medicine of this invention is one or a combination thereof selected freely from the group consisting of an immunotherapeutic agent, a cytotoxic agent, a growth inhibitory agent, a radiation therapy agent, and an anti-angiogenesis agent. The medicines which are most suitable for the treatment can be further found out through the medicine treatment.

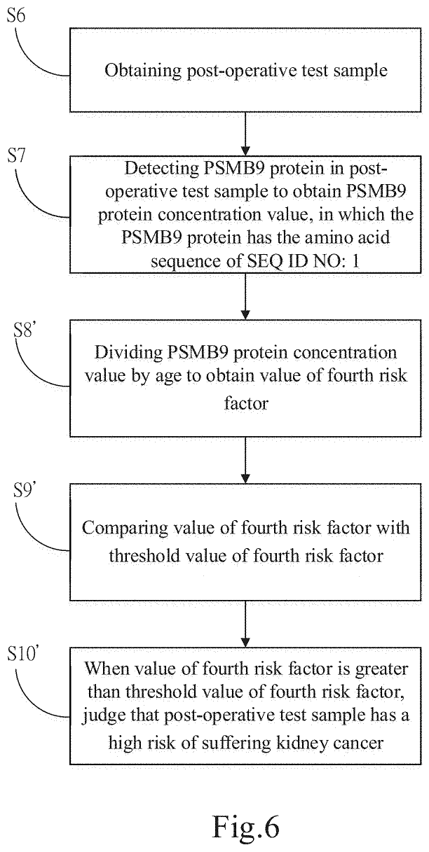

Finally, referring to FIG. 6, it is the step flowchart in the fourth embodiment of this invention. As shown in the figure, there is a method of judging the risk of suffering kidney cancer, the steps comprises:

S6: Obtaining post-operative test sample;

S7: Detecting PSMB9 proteins in post-operative test sample to obtain PSMB9 protein concentration value, in which PSMB9 protein has the amino acid sequence of SEQ ID NO: 1;

S8′: Dividing PSMB9 protein concentration value by age to obtain value of fourth risk factor;

S9′ : Comparing value of fourth risk factor with threshold value of fourth risk factor; and

S10′: When value of fourth risk factor is greater than threshold value of fourth risk factor, judge that post-operative test sample has a high risk of suffering kidney cancer.

The fourth embodiment of this Invention is used as the method of judging the risk of suffering kidney cancer after surgery; the steps are identical to the ones in the second embodiment, of which a threshold value of the fourth risk factor of the fourth embodiment is 0.00032 [(fmol/μg)/age]. Another difference is that the sample obtained in the fourth embodiment of this invention are the post-operative test sample taken from the test subject that have undergone the medicine treatment or the surgical treatment or a combination of both; the medicines are selected from one or a combination thereof selected freely from the group consisting of the immunotherapeutic agent, the cytotoxic agent, the growth inhibitory agent, the radiation therapy agent, and the anti-angiogenic agent. This method of judging the risk of suffering kidney cancer can be used to further judge the effectiveness of the medicine used by the test subject to find the medicines most suitable for treatment.

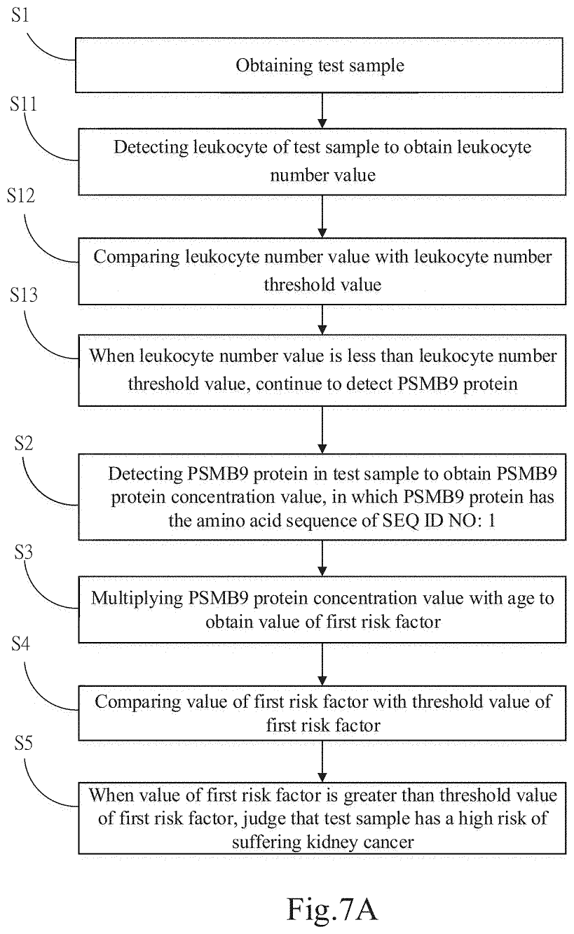

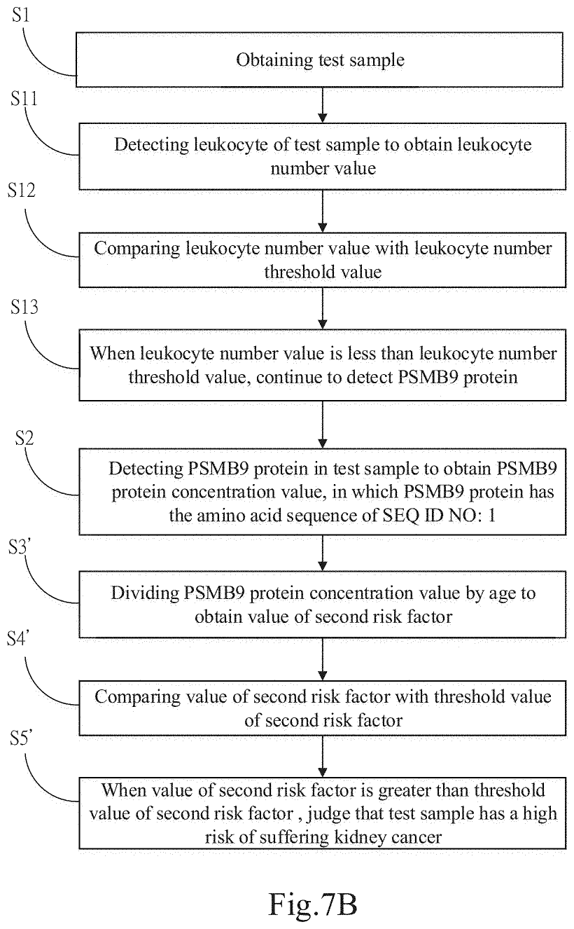

Moreover, in the first/second embodiment of this invention, in order to prevent the concentration of the PSMB9 protein from rising up due to kidney inflammation and affecting the judgment of the risk of kidney cancer, it should refer to FIG. 7A and FIG. 7B, which are the flowchart of the test steps collocating leukocytes detection in the first embodiment of this Invention, and the flowchart of the test steps collocating leukocytes detection in the second embodiment of this invention, respectively.

After the step S1 of the first/second embodiment, it further comprises the following steps:

S11: Detecting leukocyte of test sample to obtain leukocyte number value;

S12: Comparing leukocyte number value with leukocyte number threshold value; and

S13: When leukocyte number value is less than leukocyte number threshold value, continue to detect PSMB9 protein.

As shown in step S11, leukocytes in the test sample (urine) are detected to obtain a leukocyte number value, in which the leukocyte number value is obtained by a general precipitation method or a conventional detection method.

As shown in step S12, compare the leukocyte number value with a leukocyte number threshold value, in which the leukocyte number threshold value is 30/HPF. Finally, as shown in step S13, when the leukocyte number value is less than the leukocyte number threshold value, continue to detect the PSMB9 protein.

Judging whether this leukocyte number value is higher than 30/HPF or not can avoid urinary tract infection that would causes misjudgment. If the urinary tract infection causes the leukocyte number value in the urine higher than the threshold (the leukocyte number threshold value is 30/HPF), treat the urinary tract infection, making the leukocyte number value be lower than 30/HPF, and then check the PSMB9-related risk factors to accurately judge whether the patient meets the kidney cancer criteria or not.

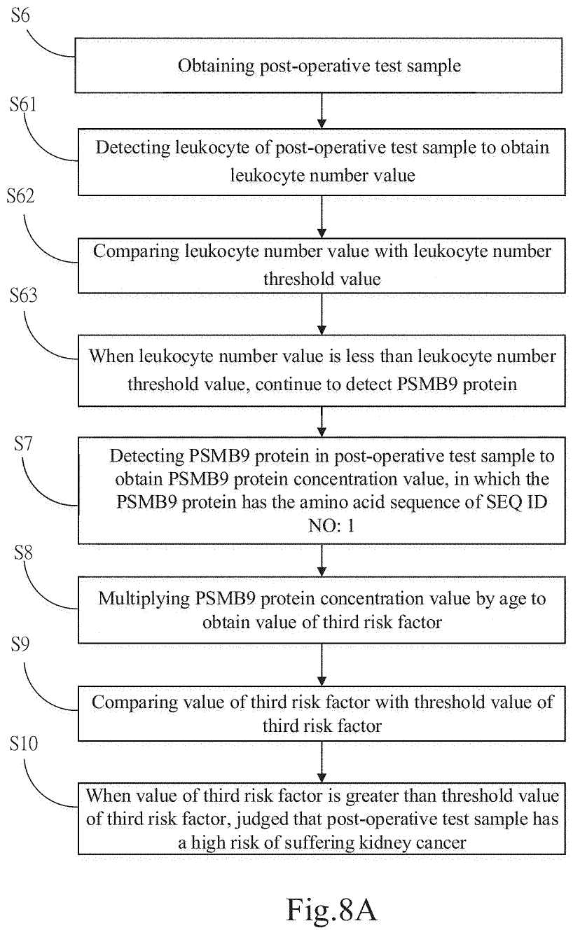

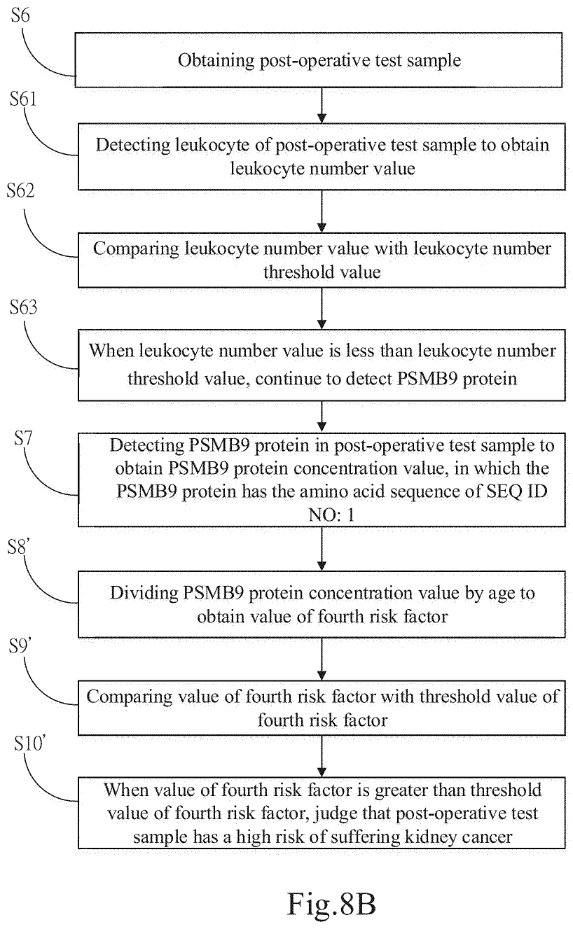

Also, referring to FIG. 8A and FIG. 8B, which are the flowchart of the test steps collocating leukocytes detection in the third embodiment of this invention, and the flowchart of the test steps collocating leukocytes detection in the fourth embodiment of this invention, respectively. Similarly, after step S6 of the third/fourth embodiment, it further comprises the following steps:

S61: Detecting leukocyte of post-operative test sample to obtain leukocyte number value;

S62: Comparing leukocyte number value with leukocyte number threshold value; and

S63: When leukocyte number value is less than leukocyte number threshold value, continue to detect PSMB9 protein.

As shown in step S61, leukocyte in the post-operative test sample (urine) is detected to obtain a leukocyte number value, wherein the leukocyte number value is obtained by a general precipitation method or a conventional detection method.

As shown in step S62, comparing the leukocyte number value with a leukocyte number threshold value, of which the leukocyte number threshold value is 30/HPF. Finally, as shown in step S63, when the leukocyte number value is less than the leukocyte number threshold value, continue to detect the PSMB9 protein.

Judging if the leukocyte number value is higher than 30/HPF or not can avoid misjudgment due to urinary tract infection. If the urinary tract infection causes the leukocyte number value in the urine higher than the threshold (the leukocyte number threshold value is 30/HPF), treat the urinary tract infection, making the leukocyte number value be lower than 30/HPF, and then check the PSMB9-related risk factors to accurately judge whether the medicines are effective in treating kidney cancer. Associating the aforesaid embodiments, we can see that in the test sample of kidney cancer cells, the expression level of PSMB9 protein was indeed significantly higher than the non-cancer cells in the test sample, and the correlation of expression level and the development stage of cancer cells can figure out; therefore, it can be used as an indicator of kidney cancer risk.

Therefore, this Invention is actually an innovative and progressive invention that can be applied in medical field, which should surely meet ROC patent application requirements patent law. We thus submit an invention patent application according to the law, expecting that the Bureau can grant the patent soon.

The statements stated above are only the preferred embodiments of this invention, which are not used to limit the claims of this invention; all the equivalent changes and modifications made according to the claims of the patent application of this invention should also belong to the claims covered in this invention.

What is claimed is:

1. A method of judging a risk of suffering kidney cancer, includes the steps of:
obtaining a test sample;
detecting a PSMB9 protein in the test sample to obtain a PSMB9 protein concentration value, in which the PSMB9 protein has the amino acid sequence of SEQ ID NO: 1;
the PSMB9 protein concentration value to obtain a value of a risk factor;
comparing the value of the risk factor with a threshold value of the risk factor; and
when the value of the risk factor is greater than the threshold value of the risk factor, judge that the test sample has a high risk of suffering kidney cancer;
wherein the threshold value of the risk factor is 0.037 (fmol PSMB9/μg urine protein).

2. A method of judging a risk of suffering kidney cancer, includes the steps of:
obtaining a test sample;
detecting a PSMB9 protein in the test sample to obtain a PSMB9 protein concentration value, in which the PSMB9 protein has the amino acid sequence of SEQ ID NO: 1;
multiplying the PSMB9 protein concentration value by an age to obtain a value of a first risk factor;
comparing the value of the first risk factor with a threshold value of the first risk factor; and when the value of the first risk factor is greater than the threshold value of the first risk factor, judge that the test sample has a high risk of suffering kidney cancer;
wherein the threshold value of the first risk factor is 2.687 (fmol/μg*age).

3. A method of judging a risk of suffering kidney cancer, includes the steps of:
obtain a test sample;
detecting a PSMB9 protein in the test sample to obtain a PSMB9 protein concentration value, in which the PSMB9 protein has the amino acid sequence of SEQ ID NO: 1;
dividing the PSMB9 protein concentration value by an age to obtain a value of a second risk factor;
comparing the value of the second risk factor with a threshold value of the second risk factor; and
when the value of the second risk factor is greater than the threshold value of the second risk factor, judge that the test sample has a high risk of suffering kidney cancer;
wherein the threshold value of the second risk factor is 0.00032 [(fmol/μg)/age].

4. The method of judging a risk of suffering kidney cancer in claim 2 or 3; in the step of obtaining a test sample, the test sample comes from the urine of a test subject.

5. The method of judging the risk of suffering kidney cancer in claim 2 or 3; in the step of obtaining a test sample, further includes the following steps of:
detecting a leukocyte of the test sample to obtain a leukocyte number value;
comparing the leukocyte number value with a leukocyte number threshold value; and
when the leukocyte number value is less than the leukocyte number threshold value, continue to detect the PSMB9 protein;
wherein the leukocyte number threshold value is 30/HPF, and the leukocyte number value is obtained by a general precipitation method or a conventional detection method.

6. A method of judging a risk of suffering kidney cancer; includes the steps of:
obtaining a post-operative test sample; the post-operative test sample is taken from a test subject that has undergone a medicine treatment or a surgical treatment or a combination of both;
detecting a PSMB9 protein in the post-operative test sample to obtain a PSMB9 protein concentration value, the PSMB9 protein has the amino acid sequence of SEQ ID NO: 1;
multiplying the PSMB9 protein concentration value by an age to obtain a value of a third risk factor;
comparing the value of the third risk factor with a threshold value of the third risk factor; and
when the value of the third risk factor is greater than the threshold value of the third risk factor, judge that the post-operative test sample has a high risk of suffering kidney cancer;
wherein the threshold value of the third risk factor is 2.687 (fmol/μg*age).

7. A method of judging a risk of suffering kidney cancer; include the steps of:
obtaining a post-operative test sample, the post-operative test sample is taken from a test subject that has undergone a medicine treatment or a surgical treatment or a combination of both;
detecting a PSMB9 protein in the post-operative test sample to obtain a PSMB9 protein concentration value; the PSMB9 protein has the amino acid sequence of SEQ ID NO: 1;
dividing the PSMB9 protein concentration value by an age to obtain a value of a fourth risk factor;
comparing the value of the fourth risk factor with a threshold value of the fourth risk factor; and
when the value of the fourth risk factor is greater than the threshold value of the fourth risk factor, judge that the post-operative test sample has a high risk of suffering kidney cancer;
wherein the threshold value of the fourth risk factor is 0.00032 [(fmol/μg)/age].

8. The method of judging a risk of suffering kidney cancer in claim 6 or 7, in the step of obtaining a post-operative test sample, the post-operative test sample is from the urine of the test subject.

9. The method of judging a risk of suffering kidney cancer in claim 6 or 7, in the step of obtaining a post-operative test sample; the post-operative test sample is taken from a test subject that has undergone a medicine treatment or a surgical treatment or a combination of both; the medicines are selected from the group consisting of the immunotherapeutic agent, the cytotoxic agent, the growth inhibitory agent, the radiation therapy agent, and the anti-angiogenic agent and a combination thereof.

10. The method of judging a risk of suffering kidney cancer in claim 6 or 7, in the step of obtaining a post-operative test sample; the post-operative test sample is taken from a test subject that has undergone a medicine treatment or a surgical treatment or a combination of both, further includes the following steps of:
detecting a leukocyte of the post-operative test sample to obtain a leukocyte number value;
comparing the leukocyte number value with a leukocyte number threshold value; and
when the leukocyte number value is less than the leukocyte number threshold value, continue to detect the PSMB9 protein;
wherein the leukocyte number threshold value is 30/HPF, and the leukocyte number value is obtained by a general precipitation method or a conventional detection method.

11. The method of judging a risk of suffering kidney cancer in claim 2 or 3 or 6 or 7, the PSMB9 protein concentration value is determined by a liquid chromatography/multiplex reaction monitoring mass spectrometer.
